(19) United States
(12) Patent Application Publication
(10) Pub. No.: US 2004/0157277 A1
(43) Pub. Date: Aug. 12, 2004

(54) Methods for predicting and/or diagnosing the risk of gastric cancer

(76) Inventors: Robert Llewellyn Clancy, Newcastle (AU); Gerald Pang, Elizabeth Bay (AU)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/695,111

(22) Filed: Oct. 28, 2003

Related U.S. Application Data
(63) Continuation of application No. 09/979,594, filed on Mar. 8, 2002.
Application No. 09/979,594 is a 371 of international application No. PCT/AU00/00441, filed on May 15, 2000.

(51) Int. Cl. G01N 33/574; G01N 33/554; G01N 33/569

(52) U.S. Cl. 435/7.23; 435/7.32

(57) ABSTRACT

The present invention relates to methods of predicting the risk of developing cancer and in particular to a method for diagnosing, and/or predicting the risk of developing gastric cancer in a subject infected with Helicobacter.

### TECHNICAL FIELD

The present invention relates to methods of predicting the risk of developing cancer and in particular to a method for diagnosing, and/or predicting the risk of developing, gastric cancer in a subject infected with Helicobacter pylori.

### BACKGROUND

Helicobacter pylori infection is now recognised as an essential pre-requisite for the development of gastric cancer. About 30% of the population become infected with this bacterium and commonly present with chronic gastritis. This may be complicated by gastric or duodenal ulceration, or may present as non-ulcer dyspepsia. A sizeable number of carriers are asymptomatic. However, in a small number of patients with H. pylori, their condition evolves through stages (including epithelial cell metaplasia and dysplasia) into neoplasia. The factors responsible for this evolution are complicated, but involve geographical, environmental and genetic parameters. Of particular importance is the host response. Current evidence supports the theory that a particular T cell response known as Th1 (characterised by production of γinterferon (γIFN) but not interleukin-4 (IL-4)) as promoting mucosal damage. Alternatively, a Th0 response can occur which includes balanced production of these cytokines (γIFN and IL-4) and which favours protection from mucosal damage. Patterns of mucosal cytokine response associated with neoplastic transformation and tumour progression have not been described.

Current Management Practice of H. pylori Infection

H. pylori is an essential component of the chain of events leading to chronic gastritis and peptic ulceration. Eradication of infection with antibiotics induces an 80-90% cure rate of peptic ulceration. A widely accepted treatment paradigm is based on detection of infection using antibody assays, followed by combination antibiotic therapy without prior endoscopic diagnosis. Endoscopy, before eradication therapy is generally accepted when ‘danger’ symptoms (eg, severe pain, bleeding) occur, or a significant risk of gastric cancer is present. However, endoscopy is a procedure which is associated with its own risks and is to be avoided if possible.

At present, no non-invasive test exists which would allow for prediction or diagnosis of gastric cancer in patients which Helicobacter infection. Such a test would be particularly valuable for patients who present with relatively mild symptoms but who are identified as being in a “high risk” category and who would otherwise automatically be required to undergo an endoscopy—with its attendant risks. Even in patients who present with “danger symptoms” and who may still require an endoscopy, such a non-invasive test could be used as a complementary tool in diagnosis. This change in practice could have a significant impact on health economics.

It is an object of the present invention to overcome or ameliorate at least one of the disadvantages of the prior art, or to provide a useful alternative.

### SUMMARY OF THE INVENTION

It has surprisingly been found that mucosal IgG2 anti-H. pylori antibody and γIFN levels are decreased and IL-4 levels are elevated in patients having Helicobacter infection when gastric cancer or precancer lesions (metaplasia and dysplasia) are present. These changes are also reflected in the blood of such patients. However, the changes are not seen in other disorders in which Helicobacter pylori is colonising the gastric mucosa.

According to a first aspect, the present invention provides a method of diagnosing and/or determining the risk of developing gastric cancer in a subject with a Helicobacter infection, including:

a) determination of IgG2 anti-H. pylori antibody level in the subject;

b) comparison of the IgG2 anti-H. pylori antibody level with a predetermined control IgG2 anti-H. pylori antibody level, wherein a reduction in the level of IgG2 anti-H. pylori antibody in the subject compared to the control indicates the presence and/or increased risk of developing gastric cancer.

According to a second aspect, the present invention provides a method of diagnosing and/or determining the risk of developing gastric cancer in a subject with a Helicobacter infection, including:

a) determination of γIFN level in the subject;

b) comparison of the γIFN level with a predetermined control γIFN level, wherein a reduction in the level of γIFN in the subject compared to the control indicates the presence and/or increased risk of developing gastric cancer.

According to a third aspect, the present invention provides a method of diagnosing and/or determining the risk of developing gastric cancer in a subject with a Helicobacter infection, including:

a) determination of IL-4 level in the subject;

b) comparison of the IL-4 level with a predetermined control IL-4 level, wherein an elevation in the level of IL-4 in the subject compared to the control indicates the presence and/or increased risk of developing gastric cancer.

According to a fourth aspect, the present invention provides a method of diagnosing and/or determining the risk of developing gastric cancer in a subject with a Helicobacter infection, including a combination of a method according to the first aspect and/or a method according to claim second aspect and/or a method according to the third aspect.

According to a fifth aspect, the present invention provides a method of diagnosing and/or determining the risk of developing gastric cancer in a subject with a Helicobacter infection, including a combination of a method according to the second aspect and a method according to the third aspect.

Preferably, the Helicobacter infection is a Helicobacter pylori infection.

Preferably, the IgG2 anti-H. pylori antibody, γIFN and/or IL-4 levels are determined by detection in a sample of biological fluid such as for example blood, saliva, gastric fluid and the like.

Preferably, the measurement of IgG2 anti-H. pylori antibody, γINF and/or IL-4 either simultaneously provides, or can be performed simultaneously with, a method which provides an indication of H. pylori status.

Preferably, the IgG2 anti-H. pylori antibody and/or γIFN and/or IL-4 are detected by a near-subject assay. The assay may, however, also be a laboratory-based test. Preferably, the assay is an antibody assay although it will be understood that other known methods of measurement can also be effectively used. Most preferably, the assay is an ELISA.

According to a sixth aspect, the present invention provides a method of predicting the risk of, and/or diagnosing, gastric cancer in a subject having a Helicobacter infection by

a) determining the frequency of IgG2 anti-H. pylori antibody- and/or γIFN- and/or IL-4-producing cells in the subject's blood; and

b) comparison of the frequency of IgG2 anti-H. pylori antibody- and/or γIFN- and/or IL-4-producing cells in the subject's blood with a predetermined control level, wherein a reduction in the level of IgG2 anti-H. pylori antibody- and/or γIFN-producing cells and/or an elevation in IL-4-producing cells in the subject's blood indicates the presence and/or increased risk of developing gastric cancer.

It will be clear to the skilled addressee that the blood may be purified to provide an enriched white blood cell population and the white blood cell population may be further fractionated to obtain specific cell populations.

Preferably, the IgG2 anti-H. pylori antibody- and/or γIFN- and/or IL-4-producing cells are stimulated with H. pylori antigen prior to measurement of IgG2 anti-H. pylori antibody and/or γIFN and/or IL-4.

According to a seventh aspect, the present invention provides a method of predicting the risk of, and/or diagnosing, gastric cancer in a subject having a Helicobacter infection by

a) determining the frequency of IgG2 anti-H. pylori antibody and/or γIFN and/or IL-4-producing cells in the subject's gastric mucosa; and

b) comparison of the frequency of IgG2 anti-H. pylori antibody and/or γIFN and/or IL-4-producing cells in the subject's gastric mucosa with a predetermined control level, wherein a reduction in the level of IgG2 anti-H. pylori antibody- and/or γIFN-producing cells and/or an elevation in IL-4-producing cells in the subject's gastric mucosa indicates the presence and/or increased risk of developing gastric cancer.

Preferably, the cells are derived from a biopsy sample.

Preferably, the IgG2 anti-H. pylori antibody- and/or γIFN- and/or IL-4-producing cells are detected by flow cytometry.

Control levels of IgG2 anti-H. pylori antibody, IL-4 and/or γIFN can be established in samples of biological fluids obtained from normal individuals, ie. those not having an established H. pylori infection, or they can be established in samples from subjects with H. pylori infection who have uncomplicated chronic gastritis or asymptomatic infection or the like. In certain cases, in which subjects are followed prospectively, control levels may be internal levels, i.e. the subject's own control levels.

The method of the present invention can also be used to diagnose and/or determine the risk of developing pre-cancer lesions such as metaplasia or dysplasia by way of measurement of IgG2 anti-H. pylori antibody, γIFN and/or IL-4.

It will be clear to the skilled addressee that ratios of IgG2 anti-H. pylori antibody, γIFN or IL-4 to other parameters such as, for example total IgG anti-H. pylori antibody may be useful as a predictor of, or in the diagnosis of, gastric precancerous or cancerous conditions, including situations in which dysplasia and metaplasia are present. Refinement of the prediction and/or diagnosis of precancerous or cancerous conditions may require that specific ratios be utilised, such as the ratio of IL-4:γIFN, IgG2:total IgG or IgG2:IgG1. However, other ratios may also be useful.

In the context of the present invention, the abbreviations “γIFN” and “IFNγ” have been used interchangably in the specification to refer to the cytokine γ interferon.

### BRIEF DESCRIPTION OF THE FIGURES

FIG. 1. Detection of IL-4 in supernatants of gastric mucosal cultures from subjects with gastric cancer or pre-cancer lesions (metaplasia or dysplasia). In uncomplicated H. pylori infection (or in benign peptic ulcers) a Th1 pattern of cytokine (eg, γINF) is found.

FIG. 2. This figure illustrates a high level of correlation between secretion of IL-4 from mucosal biopsies, and H. pylori antigen stimulated blood T cells. IL-4 was not secreted from antigen stimulated T cells in untreated subjects with uncomplicated chronic gastritis and H. pylori infection.

FIG. 3. Cytokine (IL-8, IL-4 and γIFN) production in the gastric mucosa of subjects infected with H. pylori.

FIG. 4. IgG1 and IgG2 anti-H. pylori antibody levels in serum of H. pylori-infected subjects having various gastrointestinal disorders.

FIG. 5. IgG1 and IgG2 anti-H. pylori antibody levels in serum of H. pylori-infected subjects having various gastrointestinal disorders.

### DESCRIPTION OF THE PREFERRED EMBODIMENTS

The invention will now be described in more detail with reference to non-limiting examples.

It was previously known that total IgG anti-H. pylori antibody levels in blood and gastric mucosa can be used as an indicator of H. pylori status. In the following examples, therefore, it will be understood that, while IgG anti-H. pylori can be utilised as a general indicator of H. pylori status, the invention also relates to the measurement of the IgG2 subclass which can be used as a predictor of, or in the diagnosis of, gastric cancer.

Techniques for measurement of cytokines and antibodies in human samples are well-known in the art and protocols and reagents are readily available. Examples of some of the techniques used are indicated below as an illustration of how some measurements may be performed.

Unless indicated otherwise, standard techniques which can be ascertained from standard texts and laboratory manuals may be employed.

### EXAMPLE 1

Determination of Cytokine and Antibody Levels in a Blood Sample

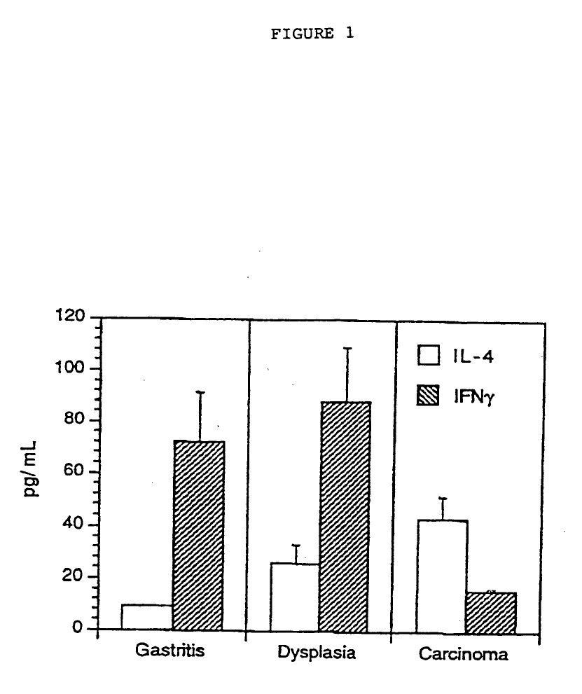

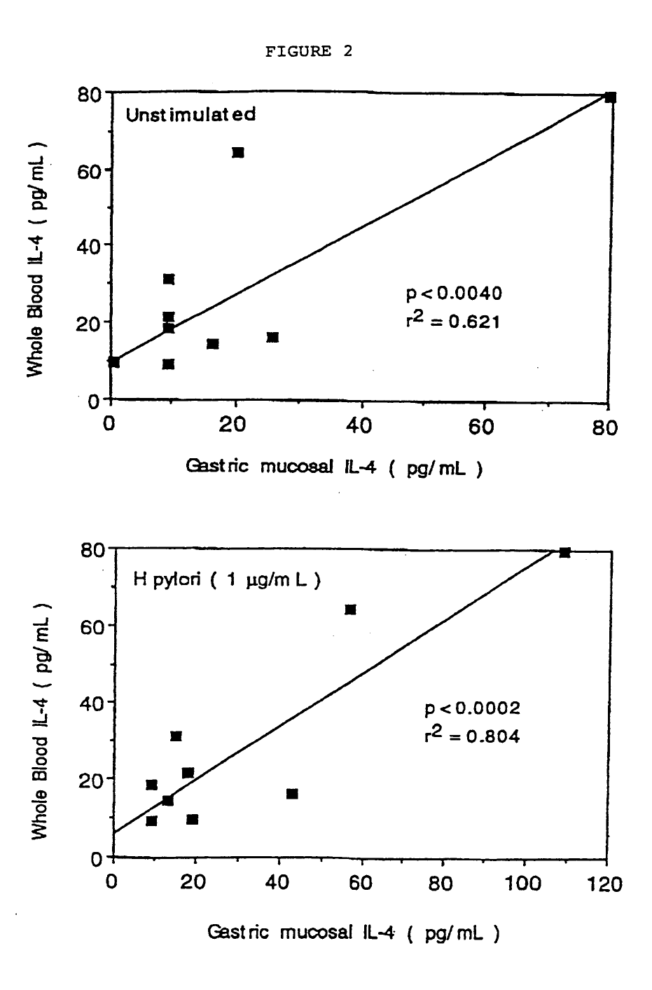

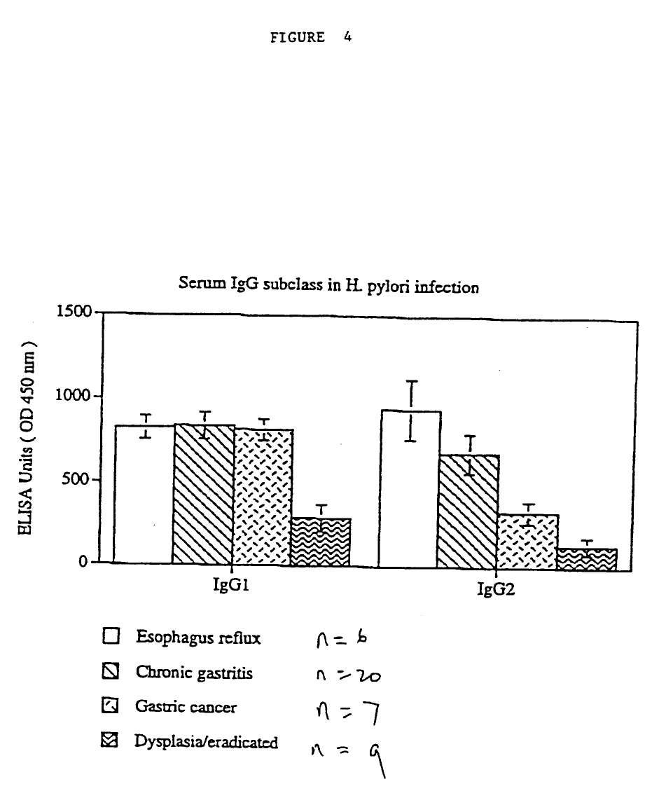

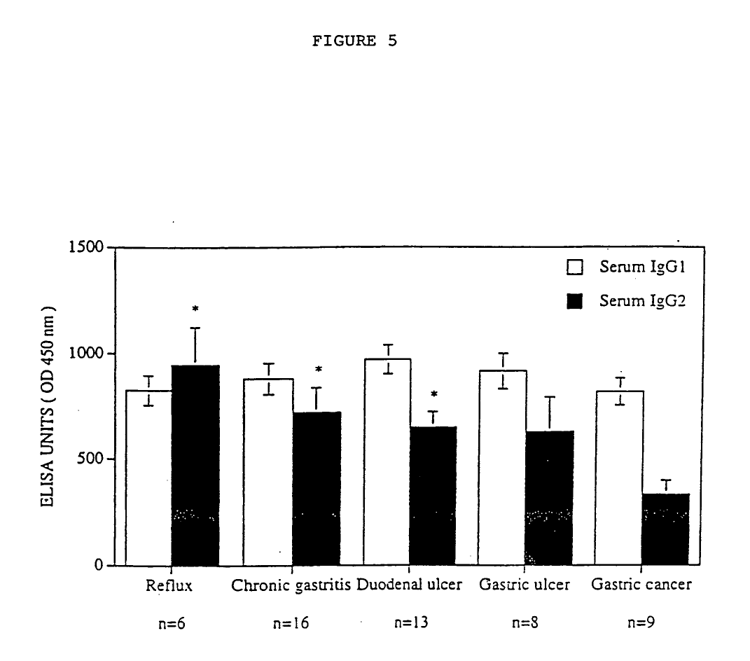

The standard assay involves coating microwells of a 96-well microtitre plate with monclonal anti-IL-4 (MoAb). After removal of antibody and washing with PBS/Tween 20, 100 uL of whole blood is added to each well containing an equal volume of AIM-V medium. After incubation for 24 hrs at 37° C., the plasma supernatant is removed for measurement of γIFN by ELISA (FIG. 1). The amount of IL-4 captured by IL-4 MoAb in each well is measured by ELISA (FIGS. 1 and 2). IgG1 and IgG2 subclass anti-H. pylori levels or IgG2/IgG ratios in serum from clotted blood or plasma supernatant (above) are measured by ELISA (FIGS. 4, 5). All samples are stored at −80° C. until assay.

Assay System for Measurement of IL-4 Alone or IL-4 and Anti-H. pylori IgG Antibodies at the Same Time.

Wells of a 96-well flat-bottomed microtitre plate are coated with 2 μg/mL of monoclonal anti-IL-4 capture antibody in sodium bicarbonate buffer pH 8.5. After removal of antibody solution, an equal volume of freshly collected whole blood is added to each well. After incubation for 24 hrs at 37° C., the plasma supernatant as removed and IL-4 bound is detected by reaction with biotinylated anti-IL-4 antibody and strepavidin-peroxidase conjugate. The amount of IL-4 is measured by colour development read in a plate reader with the appropriate standards.

On the same plate, IgG anti-H. pylori antibody is detected by adding the plasma supernatant to wells coated with 4 ug/mL of H. pylori antigens using an ELISA assay. The results are shown in Table 1.

TABLE 1
IL-4 production and anti-H. pylori IgG antibody in whole blood

| | IL-4 production | H. pylori IgG |
|---|---|---|
| Subject | (pg/mL) | (ELISA Index) |
| S1 | 42.77 | 0.696 |
| S2 | 9.4 | 1.61 |
| S3 | 13.49 | 1.86 |
| S4 | 108.25 | 0.95 |
| S5 | 9.4 | 1.83 |
| S6 | 18.1 | 0.67 |
| S7 | 9.4 | 4.32 |
| S8 | 19.41 | 3.22 |
| S9 | 56.64 | 3.48 |
| S10 | 15.1 | 3.42 |
| S11 | 9.4 | 0.12 |

### EXAMPLE 2

Frequency of IL-4 and γIFN Producing Cells in Gastric Muc sa

Gastric T cells are isolated from biopsy tissues obtained at endoscopy. The tissues is rinsed in 1 mM dithiothreitol and 1 mM EDTA to remove epithelial cells and intraepithelial cells before extraction of lamina propria T cells in serum-free AIM-V medium containing 40 U collagenase (Worthington Biochemical) for 2-3 hrs. The viability of the mononuclear cells after removal of undigested materials was >90% by trypan blue exclusion. Isolated gastric mononuclear cells from individual biopsies are usually too low (about 0.503×10⁵ cells per biopsy) for antigen-mediated re-stimulation in bulk cultures. Therefore, IL-4 and γIFN producer frequencies in each cell isolate are determined by intracellular staining and then analysed on the FACS Vantage using 3-colour flow cytometry. Isolated gastric cells were activated with PMA and ionomycin and PMA, stained with PerCP-CD3 monoclonal antibody (Becton Dickinson) and then processed for intracellular staining with FITC-γIFN and PE-IL-4 monoclonal antibody as described above.

Unless indicated otherwise above, standard techniques which can be ascertained from standard laboratory texts were used.

Table 2 provides an example of the predictions/diagnoses which can be made on the basis of the above tests.

TABLE 2

| | |
|---|---|
| IgG anti-H. pylori antibody +ve | low cancer risk |
| IL-4 −ve | endoscopy not indicated on age |
| | indications alone |
| IgG anti-H. pylori antibody +ve | high cancer risk |
| IL-4 +ve | needs endoscopy as early |
| | intervention |
| IgG anti-H. pylori antibody −ve | no evidence of H. pylori infection |
| IL-4 −ve | |

### EXAMPLE 3

Frequency of IL-4 and γIFN Producing Cells in Peripheral Blood

Intracellular cytokine staining and detection by flow cytometry is used to estimate cytokine-producer frequencies of IL-4 and γIFN amongst different subjects. This allows comparison of results obtained from gastric biopsy tissue where analysis by limiting dilution culture following antigen re-stimulation is not possible due to low numbers of cells isolated per biopsy. Peripheral blood mononuclear cells or whole blood is activated with phorbol myristate acetate (PMA, 50 ng/mL) and 1 μM ionomycin for 4-5 hrs in the presence of 2 μM monensin, fixed, permeabilised and stained with FITC/PE labelled γIFN/IL-4 (Bectin-Dickinson). γIFN and IL-4 frequencies are then analysed by flow cytometry with matched isotype IgG control and gated for lymphocytes.

The frequencies of IL-4 and γIFN producing cells in peripheral blood from subjects with or without H. pylori infection are shown in Tables 3 and 4. The ratios of γIFN:IL-4 producing cells were higher in subjects infected with H. pylori than in non-infected subjects.

Limiting dilution analysis was used to determine quantitative estimates of the frequency of circulating IL-4 and γIFN-secreting cells in blood using short-term cultures stimulated with Hp recombinant antigen (citrate synthase of Hp 0310). A non-protective recombinant antigen Hp-0162 was used as a negative control. Cells are seeded in V-bottomed 96-well microplate using twofold dilution from 10⁵ to 2.5×10³ cells at 24 replicates per cell concentration. Cultures were stimulated with a predetermined concentration of citrate synthase or Hp 0310 antigen in the presence of rIL-2 (5 U/mL) for 3 days. Controls contained no responder cells or responder cells in medium and rIL-2 without antigen. As IL-4 is unstable an antibody capture method is used with bound IL-4 measured by ELISA using a matched antibody pair (Endogen/CSL). γIFN production is measured in the supernatant by standard methods. Frequencies of peripheral blood mononuclear cells producing IL-4 and γIFN are calculated by maximum likelihood method using appropriately validated computer software.

TABLE 3
Cytokine producing cells in H. pylori antibody
POSITIVE subjects

| Subjects | γIFN(%) | IL-4 (%) | γIFN:IL-4 ratio |
|---|---|---|---|
| S1 | 18.3 | 2.5 | 7.3 |
| S2 | 25.4 | 3.3 | 7.6 |
| S3 | 26.0 | 11.5 | 2.3 |
| S4 | 9.6 | 2.6 | 3.7 |
| S5 | 14.8 | 5.6 | 2.6 |
| Mean ± SE | | | 4.7 ± 1.15* |

TABLE 4
Cytokine producing cells in H. pylori antibody
NEGATIVE subjects

| Subjects | γIFN (%) | IL-4 (%) | γIFN:IL-4 ratio |
|---|---|---|---|
| S6 | 24.8 | 28.3 | 0.9 |
| S7 | 8.9 | 3.0 | 3.0 |
| S8 | 8.1 | 2.6 | 3.1 |
| S9 | 29.9 | 29.1 | 1.0 |
| S10 | 25.9 | 17.2 | 1.5 |
| Mean ± SE | | | 1.9 ± 0.48* |

*p = 0.054

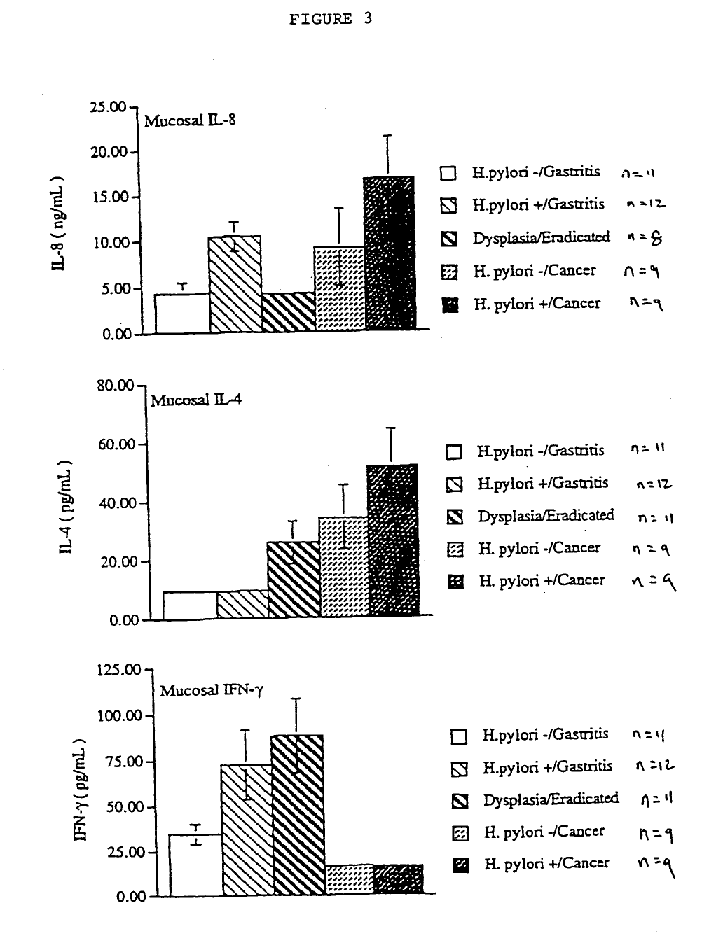

FIGS. 1 to 5 provide results obtained utilising the tests exemplified below in studies of subjects having various gastrointestinal conditions i.e. reflux, gastritis, duodenal ulcer, gastic ulcer and gastric cancer. The Figures are self-explanatory and show that levels of IgG2, γIFN and IL-4 can be used as predictors of, and in the diagnosis of, gastric cancer in patients having H. pylori infection.

Although the invention has been described with reference to specific examples, it will be appreciated by those skilled in the art that the invention may be embodied in many other forms without departing from the spirit or intent of the inventive concept.

The claims defining the invention are as follows:

1. A method of diagnosing and/or determining the risk of developing gastric cancer in a subject with a Helicobacter infection, including:
a) determination of IgG2 anti-H. pylori antibody level in the subject;
b) comparison of the IgG2 anti-H. pylori antibody level with a predetermined control IgG2 anti-H. pylori antibody level, wherein a reduction in the level of IgG2 anti-H. pylori antibody in the subject compared to the control indicates the presence and/or increased risk of developing gastric cancer.

2. A method of diagnosing and/or determining the risk of developing gastric cancer in a subject with a Helicobacter infection, including:
a) determination of γIFN level in the subject;
b) comparison of the γIFN level with a predetermined control γIFN level, wherein a reduction in the level of γIFN in the subject compared to the control indicates the presence and/or increased risk of developing gastric cancer.

3. A method of diagnosing and/or determining the risk of developing gastric cancer in a subject with a Helicobacter infection, including:
a) determination of IL-4 level in the subject;
b) comparison of the IL-4 level with a predetermined control IL-4 level, wherein an elevation in the level of IL-4 in the subject compared to the control indicates the presence and/or increased risk of developing gastric cancer.

4. A method of diagnosing and/or determining the risk of developing gastric cancer in a subject with a Helicobacter infection, including a combination of a method according to claim 1 and/or a method according to claim 2 and/or a method according to claim 3.

5. A method of diagnosing and/or determining the risk of developing gastric cancer in a subject with a Helicobacter infection, including a combination of a method according to claim 2 and a method according to claim 3.

6. A method according to any one of claims 1 to 6 wherein the Helicobacter infection is a Helicobacter pylori infection.

7. A method according to any one of claims 1 to 7 wherein the IgG2 anti-H. pylori antibody, γIFN and/or IL-4 levels are determined by detection of the levels in a sample of biological fluid.

8. A method according to claim 7 wherein the biological fluid is blood.

9. A method according to claim 7 wherein the biological fluid is saliva.

10. A method according to claim 7 wherein the biological fluid is gastric fluid.

11. A method according to any one of claims 1 to 10 wherein the measurement of IgG2 anti-H. pylori antibody, γINF and/or IL-4 either simultaneously provides, or can be performed simultaneously with, a method which provides an indication of H. pylori status.

12. A method according to any one of claims 1 to 11 wherein the IgG2 anti-H. pylori antibody, γIFN and/or IL-4 are detected by a near-subject assay.

13. A method according to any one of claims 1 to 11 wherein the assay is a laboratory-based test.

14. A method according to claim 12 or claim 13 wherein the assay is an antibody assay.

15. A method according to claim 14 wherein the antibody assay is an ELISA.

16. A method of predicting the risk of, and/or diagnosing, gastric cancer in a subject having a Helicobacter infection by
a) determining the frequency of IgG2 anti-H. pylori antibody- and/or γIFN- and/or IL-4-producing cells in the subject's blood; and
b) comparison of the frequency of IgG2 anti-H. pylori antibody- and/or γIFN- and/or IL-4-producing cells in the subject's blood with a predetermined control level, wherein a reduction in the level of IgG2 anti-H. pylori antibody- and/or γIFN-producing cells and/or an elevation in IL-4-producing cells in the subject's blood indicates the presence and/or increased risk of developing gastric cancer.

17. A method according to claim 16 wherein the blood is purified to provide an enriched white blood cell population.

18. A method according to claim 17 wherein the white blood cell population is further fractionated to obtain specific cell populations.

19. A method according to any one of claims 16 to 18 wherein the IgG2 anti-H. pylori antibody- and/or γIFN- and/or IL-4-producing cells are stimulated with H. pylori antigen prior to measurement of IgG2 anti-H. pylori antibody and/or γIFN and/or IL-4.

20. A method of predicting the risk of, and/or diagnosing, gastric cancer in a subject having a Helicobacter infection by
a) determining the frequency of IgG2 anti-H. pylori antibody and/or γIFN and/or IL-4-producing cells in the subject's gastric mucosa; and
b) comparison of the frequency of IgG2 anti-H. pylori antibody and/or γIFN and/or IL-4-producing cells in the subject's gastric mucosa with a predetermined control level, wherein a reduction in the level of IgG2 anti-H. pylori antibody- and/or γIFN-producing cells and/or an elevation in IL-4-producing cells in the subject's gastric mucosa indicates the presence and/or increased risk of developing gastric cancer.

21. A method according to claim 20 wherein the cells are derived from a biopsy sample.

22. A method according to claim 20 or claim 21 wherein of IgG2 anti-H. pylori antibody and/or γIFN and/or IL-4-producing cells are detected by flow cytometry.
